(19) United States
(12) Patent Application Publication
(10) Pub. No.: US 2022/0395219 A1
(43) Pub. Date: Dec. 15, 2022

(54) SKIN STATE DETERMINATION METHOD AND SKIN STATE DETERMINATION SYSTEM

(71) Applicant: Panasonic Intellectual Property Management Co., Ltd., Osaka (JP)

(72) Inventors: MIZUHO SAKAKIBARA, Tokyo (JP); NAOMI TOMIYAMA, Kyoto (JP); ATSUSHI SASO, Osaka (JP); JUNICHI FUJIHIRA, Kanagawa (JP); MIKIKO MATSUO, Nara (JP); YUICHI AOKI, Osaka (JP); MOTOJI OHMORI, Osaka (JP)

(21) Appl. No.: 17/821,522

(22) Filed: Aug. 23, 2022

Related U.S. Application Data
(63) Continuation of application No. PCT/JP2021/005396, filed on Feb. 15, 2021.

(30) Foreign Application Priority Data
Mar. 18, 2020 (JP) 2020-048322

(51) Int. Cl. A61B 5/00

(52) U.S. Cl. CPC A61B 5/443; A61B 5/0075; A61B 5/0077; A61B 5/0035; A61B 5/486; A61B 5/444; A61B 5/7264; A61B 5/6898; A61B 2560/0247

(57) ABSTRACT

A skin state determination method includes: acquiring a first measurement result including data obtained by a spectral camera measuring the skin of a user at a first time point, second time points before the first time point being different from each other; estimating, on the basis of the first measurement result, a first estimation result including at least one of a skin moisture content or hidden blemish conditions of the user at the first time point; and determining a skin state of the user on the basis of the first estimation result and second estimation results based on second measurement results, each of the second measurement results including data obtained by the spectral camera measuring the skin of the user at each of the second time points.

### BACKGROUND

### 1. Technical Field

The present disclosure relates to a skin state determination method and a skin state determination system.

### 2. Description of the Related Art

In the related art, skin moisture content is measured to determine a skin state (such as whether the skin is dry, for example). Skin moisture content may vary depending on weather conditions such as the temperature and humidity. Accordingly, Japanese Unexamined Patent Application Publication No. 2012-95825 discloses a technology for calculating a correlation between skin moisture content and weather conditions.

### SUMMARY

Incidentally, when determining a skin state of a user on the basis of the skin moisture content or the like, it is desirable to determine the skin state according to the user.

One non-limiting and exemplary embodiment provides a skin state determination method and a skin state determination system that can determine the skin state according to the user.

In one general aspect, the techniques disclosed here feature a skin state determination method including: acquiring a first measurement result including data obtained by a spectral camera measuring the skin of a user at a first time point, second time points before the first time point being different from each other; estimating, on the basis of the first measurement result, a first estimation result including at least one of a skin moisture content or hidden blemish conditions of the user at the first time point; and determining a skin state of the user on the basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

According to the skin state determination method and the like according to aspects of the present disclosure, the skin state according to the user can be determined.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating a schematic configuration of a skin state determination system according to an embodiment;

FIG. 2 is a block diagram illustrating a functional configuration of the skin state determination system according to the embodiment;

FIG. 3 is a sequence diagram illustrating operations by the skin state determination system according to the embodiment;

FIG. 4A is a diagram illustrating a first example of state information displayed on an information terminal according to the embodiment;

FIG. 4B is a diagram illustrating a second example of state information displayed on an information terminal according to the embodiment;

FIG. 5 is a flowchart illustrating operations by the information terminal according to the embodiment;

FIG. 6 is a flowchart illustrating operations by the server according to the embodiment;

FIG. 7 is a flowchart illustrating a first example of the operations for determining the skin state of a user illustrated in FIG. 6;

FIG. 8 is a flowchart illustrating a second example of the operations for determining the skin state of a user illustrated in FIG. 6;

FIG. 9 is a flowchart illustrating a third example of the operations for determining the skin state of a user illustrated in FIG. 6;

FIG. 10 is a flowchart illustrating a fourth example of the operations for determining the skin state of a user illustrated in FIG. 6;

FIG. 11 is a flowchart illustrating operations for controlling equipment indoors by the server according to the embodiment; and

FIG. 12 is a diagram illustrating a configuration of an information terminal according to another embodiment.

### DETAILED DESCRIPTIONS

### Overview of Present Disclosure

As described in Patent Literature 1, by calculating a correlation between skin moisture content and weather conditions, skin moisture content values measured under different weather conditions can be corrected to skin moisture content values measured under the same weather conditions, for example. In other words, two skin moisture content values can be treated as values measured under the same weather conditions. If a skin moisture content threshold for determining the skin state under prescribed weather conditions is set, a determination using the threshold is possible by correcting the skin moisture content measured under weather conditions other than the prescribed weather conditions on the basis of the correlation. With this arrangement, the skin state can be determined accurately even if the skin moisture content is measured under weather conditions different from the prescribed weather conditions.

Incidentally, the normal skin state is different for each user. For this reason, if the skin state is determined using a uniform threshold or the like, a determination appropriate to the user may not be achieved in some cases. Accordingly, the inventors thoroughly investigated skin state determination methods and the like with which the skin state according to the user can be determined, and arrived at the skin state determination method and the like described below.

A skin state determination method according to an aspect of the present disclosure includes: acquiring a first measurement result including data obtained by a spectral camera measuring the skin of a user at a first time point, second time points before the first time point being different from each other; estimating, on the basis of the first measurement result, a first estimation result including at least one of a skin moisture content or hidden blemish conditions of the user at the first time point; and determining a skin state of the user on the basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

With this arrangement, the skin state of the user can be determined on the basis of second estimation results for the user from the past. The second estimation results include at least one of the skin moisture content and hidden blemish conditions of a user U under normal conditions, for example, and are different results for each user. Therefore, by using such second estimation results, the skin state according to the user can be determined compared to the case of using a fixed threshold.

For example, in the determining, two or more second estimation results including a measurement conditions logs may be extracted as the second estimation results, and the determination may be made on a basis of the extracted two or more second estimation results, a measurement conditions log indicating measurement conditions at the first time point being identical or similar to each of the measurement conditions logs.

This arrangement makes it possible to reduce how much differences among the measurement conditions logs influence the estimation results in cases where such influence occurs. Therefore, the skin state according to the measurement conditions logs when measurement is performed at the first time point can be determined.

For example, the first estimation result may include at least the hidden blemish conditions, the skin state determination method may further include estimating an ultraviolet light intensity at the first time point on the basis of the first estimation result, and in the determining, the hidden blemish conditions of the user at a third time point after the first time point may be estimated on the basis of the hidden blemish conditions of the user based on the first estimation result and the ultraviolet light intensity, and a determination for the third time point may be made.

With this arrangement, the user is able to learn of hidden blemish conditions in the future. For example, the degree of change in the skin state (the degree of change in hidden blemishes) with respect to the ultraviolet light intensity is different for each user. If the skin state of the user at the third time point is determined on the basis of a correlation between the ultraviolet light intensity and the degree of change in the skin state for the user, the skin state according to the user can also be determined at the third time point farther in the future than the present.

For example, the skin state estimation method may further include: acquiring image data obtained by an imaging camera capturing the skin of the user; and outputting state information reflecting, in the image data, a determination result regarding the skin state of the user.

With this arrangement, the skin state can be presented to the user in an easily understood way.

For example, the state information may include information indicating an action to be recommended to the user based on the determination result.

With this arrangement, the user can learn of a recommended action by checking the state information, thereby improving convenience for the user.

For example, the spectral camera and the imaging camera may operate synchronously.

With this arrangement, the image data and the first measurement can be acquired at the same timing.

For example, the skin state determination method may further include: determining whether the user is indoors at the first time point; and if the user is determined to be indoors, controlling operations by equipment indoors according to the skin state of the user.

With this arrangement, a spatial environment according to the skin state of the user can be realized automatically. For this reason, convenience is improved further for the user.

For example, the first measurement result may include data obtained by the spectral camera measuring sunlight at the first time point, and in the estimating, the difference between the data obtained by measuring the skin of the user and the data obtained by measuring the sunlight may be taken.

With this arrangement, by removing the influence due to ambient light, the skin moisture content of the user can be estimated more accurately.

A skin state determination method according to an aspect of the present disclosure includes: acquiring a first estimation result including at least one of a skin moisture content or hidden blemish conditions of a user at a first time point, the first estimation result being based on a first measurement result including data obtained by a spectral camera measuring the skin of the user at the first time point, second time points before the first time point being different from each other; and determining a skin state of the user on the basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

With this arrangement, the acquired first estimation result can be used to determine the skin state according to the user.

A skin state determination method according to an aspect of the present disclosure includes: acquiring a first measurement result including data obtained by a spectral camera measuring the skin of a user at a first time point, second time points before the first time point being different from each other; acquiring second measurement results including data obtained by the spectral camera measuring the skin of the user at each of the second time points; and acquiring a determination result regarding a skin state of the user at the first time point by inputting the first measurement result into a trained model that has been trained to accept the second measurement results as input and output a determination result regarding the skin state of the user.

With this arrangement, a determination result regarding the skin state of the user can be acquired without estimating the skin state of the user at the first time point. Therefore, the amount of processing for determining the skin state of the user can be reduced. For example, a process for estimating the skin state at the first time point can be skipped.

The trained model may be trained to accept the second measurement results including a measurement conditions log at each of the second time points as input and output a determination result regarding the skin state of the user, and in the acquiring of a determination result, a determination result regarding the skin state of the user at the first time point may be acquired by inputting, into the trained model, the first measurement result including a measurement conditions log for the user at the first time point.

This arrangement makes it possible to reduce how much differences among the measurement conditions logs influence the estimation results in cases where such influence occurs. Therefore, the skin state according to the measurement conditions logs when measurement is performed at the first time point can be determined.

A skin state determination system according to an aspect of the present disclosure includes: an acquirer that acquires a first measurement result including data obtained by a spectral camera measuring the skin of a user at a first time point; an estimator that produces, on a basis of the first measurement result, a first estimation result including at least one of a skin moisture content or hidden blemish conditions of the user at the first time point; and a determiner that determines a skin state of the user on a basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

With this arrangement, effects similar to the skin determination method above are attained.

Note that these general or specific aspects may also be realized by a system, a method, an integrated circuit, a computer program, a non-transitory computer-readable recording medium such as a CD-ROM disc, or any selective combination thereof. The program may be pre-stored in the recording medium or supplied to the recording medium over a wide-area communication network such as the Internet.

Hereinafter, embodiments will be described specifically with reference to the drawings.

Note that the embodiments and modifications described hereinafter all illustrate general or specific examples. Features such as numerical values, shapes, materials, components, layout positions and connection states of components, steps, and the ordering of steps indicated in the following embodiments and modifications are merely examples, and are not intended to limit the present disclosure. Among the components in the following embodiments, components that are not indicated in the independent claims are described as optional components.

Note that each diagram is a schematic diagram, and does not necessarily illustrate a strict representation. In the drawings, components that are substantially the same are denoted with the same signs, and duplicate description of such components may be reduced or omitted in some cases.

In this specification, numerical values are not expressions that express only a strict meaning, but rather expressions that are meant to include substantially equivalent ranges, even differences of a few percent, for example.

### Embodiments

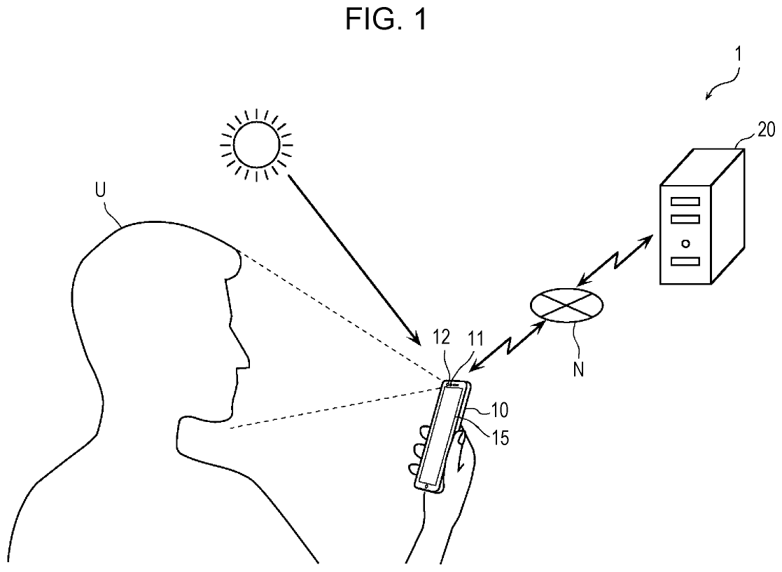

Hereinafter, a skin state determination system and the like according to the present embodiment will be described with reference to FIGS. 1 to 11.

### [1. Configuration of Skin State Determination System]

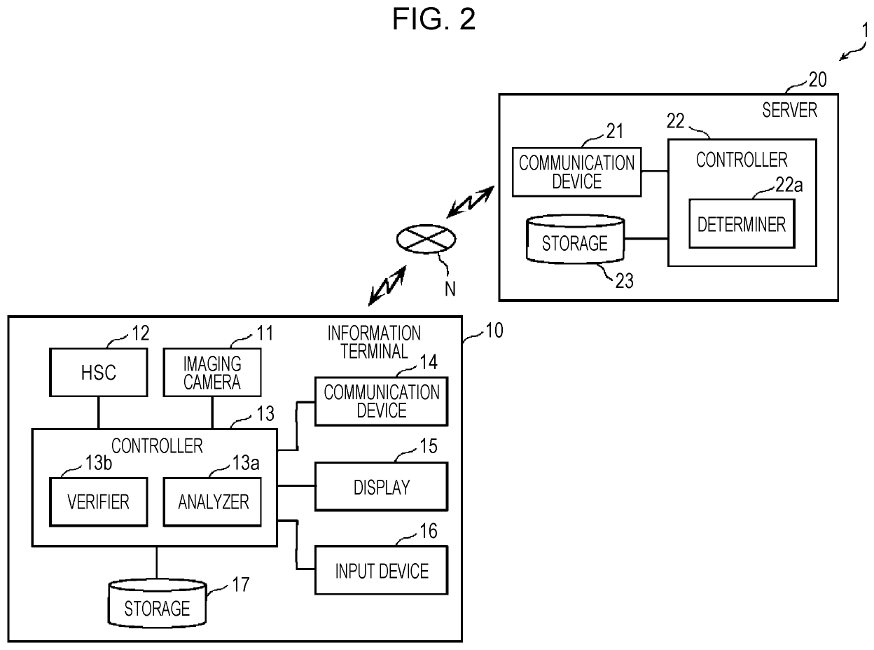

First, a configuration of a skin state determination system according to the present embodiment will be described with reference to FIGS. 1 and 2. FIG. 1 is a diagram illustrating a schematic configuration of a skin state determination system 1 according to the present embodiment. FIG. 2 is a block diagram illustrating a functional configuration of the skin state determination system 1 according to the present embodiment.

As illustrated in FIGS. 1 and 2, the skin state determination system 1 is provided with an information terminal 10 carried by a user U and a server 20. The information terminal 10 and the server 20 are communicably connected over a network N.

The information terminal 10 is a mobile device carried by the user U, and may be a smartphone or a tablet, for example. The information terminal 10 measures the spectrum of skin for determining the skin state of the user U. For example, the information terminal 10 includes an imaging camera 11, a hyperspectral camera 12, a controller 13, a communication device 14, a display 15, an input device 16, and storage 17. Note that the hyperspectral camera 12 may also be referred to as the HSC in some cases.

The imaging camera 11 captures image data. For example, the imaging camera 11 captures face image data for face recognition. The imaging camera 11 may capture face image data of the user U when the user is looking at the display 15, for example. Note that the face image data may also be used when a determination result regarding the skin state of the user U in the server 20 described later is displayed on the display 15 of the information terminal 10. The face image data is an example of image data.

The imaging camera 11 includes a lens, an image sensor such as a charge-coupled device (CCD) sensor or a complementary metal-oxide-semiconductor (CMOS) sensor, and a signal processor. For example, the imaging camera 11 combines three images of red, green, and blue obtained by forming images on the CCD or CMOS image sensor through the lens to generate a single image (color image). Note that the imaging camera 11 may also be a monochrome camera that generates a monochrome image.

The hyperspectral camera 12 is a camera that can measure the luminance for individual wavelength bands of light, and measures the spectrum of the skin of the user U and the spectrum of the ambient light around the user U. The measurement data (light spectrum data) from the hyperspectral camera 12 is used to determine the skin state of the user U. Note that the ambient light around the user U is the light that the user U is exposed to, and is sunlight, for example. Note that the spectrum of the ambient light is acquired by directly measuring sunlight, for example, but is not limited thereto.

The hyperspectral camera 12 is configured to measure light with a wavelength from 280 nm to 2500 nm, for example, but may also be configured to measure light with a wavelength from 400 nm to 2500 nm. In other words, the hyperspectral camera 12 may be capable of measurement from the ultraviolet region to the short-wave infrared region or measurement from the visible region to the short-wave infrared region.

The hyperspectral camera 12 is a camera that can acquire spectra in dozens of bands (for example, 10 bands) or more. For example, the hyperspectral camera 12 separates incident light into dozens of bands or more to acquire an image for each band.

For example, the hyperspectral camera 12 includes optics for separating incident light, a hyperspectral sensor that can acquire spectra in dozens of bands or more simultaneously, and a signal processor that generates a two-dimensional image for each band on the basis of electrical signals from the hyperspectral sensor. The optics include a slit, a diffraction grating, a lens, and the like.

For example, the hyperspectral camera 12 measures the spectrum of the skin at different positions while scanning the face of the user U, but is not limited thereto. The hyperspectral camera 12 is an example of a spectral camera.

Note that, as illustrated in FIG. 1, the imaging camera 11 and the hyperspectral camera 12 are provided on the same surface of the information terminal 10, and are provided on the same surface as the display 15, for example. With this arrangement, the imaging camera 11 and the hyperspectral camera 12 can capture or measure the face of the user U and the like when the user U is looking at the display 15. The imaging camera 11 and the hyperspectral camera 12 may also be disposed nearby on the surface. The imaging camera 11 and the hyperspectral camera 12 may be disposed side by side, for example.

The controller 13 is a control device that controls the components of the information terminal 10. The controller 13 controls the imaging camera 11 to capture face image data of the user U. The controller 13 controls the hyperspectral camera 12 to measure at least the spectrum of the skin of the user U. For example, the controller 13 may control the operations by the hyperspectral camera 12 in association with the operations by the imaging camera 11. For example, the controller 13 may control the imaging camera 11 and the hyperspectral camera 12 to operate synchronously. For example, the controller 13 may activate the hyperspectral camera 12 when the imaging camera 11 is active and cause the hyperspectral camera 12 to take a measurement. For example, the controller 13 may treat the activation of the imaging camera 11 as a trigger for activating the hyperspectral camera 12. For example, the controller 13 may treat the stopping of the imaging camera 11 as a trigger for stopping the hyperspectral camera 12. Note that the controller 13 functions as an acquirer that acquires, from the hyperspectral camera 12, measurement data obtained by measuring the skin of the user U.

The controller 13 performs various processes on the basis of the face image data acquired from the imaging camera 11 and the measurement data acquired from the hyperspectral camera 12. The controller 13 includes an analyzer 13a and a verifier 13b.

The analyzer 13a estimates at least one of a skin moisture content or hidden blemish conditions of the user U at present on the basis of a first measurement result including the measurement data acquired by the hyperspectral camera 12. In the case of estimating hidden blemish conditions, the analyzer 13a may also estimate the ultraviolet light intensity the user U is exposed to. The analyzer 13a may also be considered to perform a spectral analysis of the skin. The present is an example of a first time point.

Note that a hidden blemish is a latent blemish beneath the surface of the skin that is invisible to the eye, and may also be referred to as an incipient blemish. If a blemish is exposed to large amounts of ultraviolet light while in the hidden state, the hidden blemish will gradually rise to the surface and become visible. The ultraviolet light intensity is the amount of ultraviolet light that the user U is inferred to be exposed to. Note that the analyzer 13a is an example of an estimator.

The verifier 13b compares the face image data of the user U captured by the imaging camera 11 to face image data for verification stored in the storage 17, and determines whether the user U is a registered user.

For example, the controller 13 outputs an estimation result estimated by the analyzer 13a to the server 20 through the communication device 14. The estimation result includes identification information unique to the information terminal 10 or the user U, for example.

The communication device 14 is a communication circuit (communication module) with which the information terminal 10 communicates with the server 20 over the network N such as the Internet.

Note that the method of communication between the information terminal 10 and the server 20 is not particularly limited.

The display 15 displays various information to the user U under control by the controller 13. For example, the display 15 displays information indicating a determination result regarding the skin state of the user U. The display 15 is realized by a liquid crystal display (LCD) panel, but may also be realized by another type of display panel, such as an organic light-emitting diode (OLED) panel. The display 15 may also include a backlight.

Note that instead of or in addition to the display 15, the information terminal 10 may also present information indicating a determination result to the user U through sound.

The input device 16 is a user interface that accepts input from the user. The input device 16 is realized by one or more hardware keys (hardware buttons), sliding switches, a touch panel, or the like. The input device 16 may also accept input from the user U through speech or gestures, for example.

The storage 17 is a storage device that stores various information to be processed by the controller 13. For example, the storage 17 may store face image data to be used for verification by the verifier 13b. For example, the storage 17 may store at least one of identification information unique to the information terminal 10 or identification information unique to the user U. The storage 17 may also store at least one of face image data captured by the imaging camera 11 or measurement data measured by the hyperspectral camera 12. In other words, the storage 17 may store data (raw data) acquired from the imaging camera 11 and the hyperspectral camera 12.

Note that the information terminal 10 may be further provided with various sensors, such as a temperature and humidity sensor for measuring temperature and humidity, a gyro sensor for measuring the rotation and attitude of the information terminal 10, and a GPS sensor that acquires the location of the information terminal 10. At least one of these sensors may also be connected to the information terminal 10 though a connection terminal as an externally attached sensor.

The server 20 determines the skin state of the user U on the basis of information acquired from the information terminal 10. The server 20 includes a communication device 21, a controller 22, and storage 23.

The communication device 21 is a communication circuit (communication module) with which the server 20 communicates with the information terminal 10 over the network N such as the Internet.

The controller 22 is a control device that controls the components of the server 20. The controller 22 stores, in the storage 23, estimation results acquired from the information terminal 10 regarding the skin moisture content and hidden blemish conditions of the user U and the ultraviolet light intensity that the user U is exposed to at present. In other words, the controller 22 accumulates estimation results regarding the skin moisture content, hidden blemish conditions, and ultraviolet light intensity of the user U at present. The controller 22 accumulates the estimation results in the storage 23 in association with identification information of the information terminal 10 or identification information of the user U. With this arrangement, time series data of estimation results associated with the information terminal 10 or the user U is stored in the storage 23.

The controller 22 includes a determiner 22a. The determiner 22a determines the skin state of the user U. For example, the determiner 22a determines the skin state of the user U on the basis of the estimation result (first estimation result) acquired at present and time series data (second estimation results) of estimation results acquired at different time points before the present. For example, the determiner 22a determines the skin state of the user U at present by making a determination on the basis of the estimation result acquired at present and time series data of estimation results acquired at different time points before the present. In other words, the determiner 22a does not use a fixed threshold to determine the skin state of the user U. Note that the different time points before the present are an example of second time points.

The determiner 22a may also set a recommend action to be recommended to the user U on the basis of the determination result. For example, if the determination result includes an indication of insufficient skin moisture content, the determiner 22a may recommend rehydration. The determiner 22a may set a recommended action on the basis of a table associating determination results with recommended actions, for example.

The storage 23 is a storage device that stores various information to be processed by the controller 22. The storage 23 stores time series data of estimation results. The storage 23 stores, for each information terminal 10 or each of multiple users U, estimation results about each user U acquired from the users U. The storage 23 may also store information a correlation between at least one of the temperature or humidity around the user U and the skin moisture content of the user U. The storage 23 may also store information related to equipment corresponding to the user U and installed indoors. The equipment corresponding to the user U is electrical equipment installed in the home or office of the user U, for example, and is an air conditioner, a humidifier, a dehumidifier, a heater, or a motorized curtain, for example. Note that the storage 23 is realized by a semiconductor memory, for example.

As above, the information terminal 10 includes the hyperspectral camera 12 for measuring the spectrum of the skin of the user U and the ambient light around the user U. With this arrangement, the information terminal 10 can estimate properties such as the skin moisture content of the user U.

As above, the server 20 does not determine an estimation result about the user at present with reference to a fixed threshold, but rather on the basis of time series data of estimation results about the user in the past. With this arrangement, the server 20 can determine the skin state of each user U on the basis of the normal skin moisture content, hidden blemish conditions, and ultraviolet light intensity for each user U, and therefore can determine the skin state according to the user U.

### [2. Operations by Skin State Determination System]

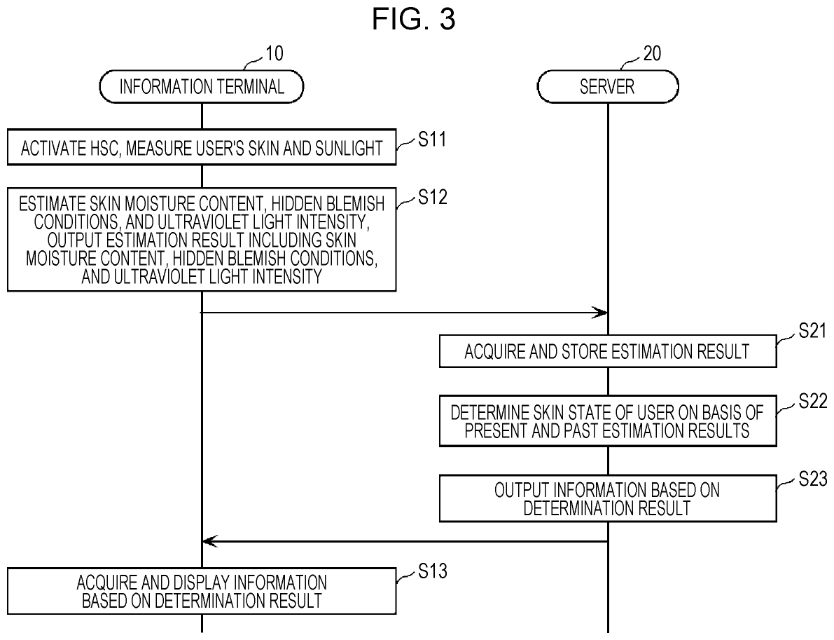

Next, operations by the above skin state determination system 1 will be described with reference to FIGS. 3 to 11. FIG. 3 is a sequence diagram illustrating operations by the skin state determination system 1 according to the present embodiment.

As illustrated in FIG. 3, the controller 13 of the information terminal 10 activates the hyperspectral camera 12 (HSC) and measures the skin of the user U and sunlight (S11). With this arrangement, a spectrum of the skin of the user U and a spectrum of sunlight can be acquired. Note that the skin measurement and the sunlight measurement may be performed at the same time. In other words, light reflected from skin and sunlight may be measured with a single measurement. For example, if it is possible to measure light reflected from skin and sunlight at the same time depending the direction of the hyperspectral camera 12 based on the time period when measurement is performed, the positions of the user U and the sun, the attitude of the information terminal 10, and the like, the spectrum of the skin of the user U and the spectrum of sunlight are measured at the same time.

The skin measurement and the sunlight measurement may also be performed at different timings. For example, after performing the skin measurement, the controller 13 may cause the display 15 to display information instructing the user U to point the hyperspectral camera 12 at the sun, and if it is detected that the hyperspectral camera 12 is pointed in the direction of the sun, the controller 13 may control the hyperspectral camera 12 to perform the sunlight measurement.

When the hyperspectral camera 12 scans the face of the user U, the controller 13 may calculate a face area of the user U with respect to the information terminal 10 on the basis of face image data captured by the imaging camera 11, and cause the calculated face area to be scanned. With this arrangement, the spectrum of the skin on the face of the user U can be acquired efficiently. The controller 13 may also calculate a face area of the user U with respect to the information terminal 10 on the basis of face image data captured by the imaging camera 11, and perform the sunlight measurement by scanning outside the calculated face area.

Note that step S11 is an example of a first acquiring step.

Next, the analyzer 13a of the controller 13 estimates the skin moisture content, hidden blemish conditions, and ultraviolet light intensity on the basis of the measurement data, and outputs an estimation result including the estimated skin moisture content, hidden blemish conditions, and ultraviolet light intensity to the server 20 (S12). The estimation of the skin moisture content, hidden blemish conditions, and ultraviolet light intensity will be described later.

Note that in step S12, it is sufficient if the analyzer 13a estimates at least one of the skin moisture content or hidden blemish conditions. In the case where hidden blemish conditions are estimated, the analyzer 13a may also estimate the ultraviolet light intensity in step S12. Step S12 is an example of a first estimating step and a second estimating step.

Next, the server 20 acquires the estimation result from the information terminal 10 and stores the acquired estimation result in association with the user U in the storage 23 (S21).

Next, the server 20 determines the skin state of the user U on the basis of the present and past estimation results (S22). The server 20 uses past estimation results to determine the skin state of the user U. The determination of the skin state will be described later. Step S22 is an example of a first determining step. Note that the past estimation results are estimated on the basis of measurement data obtained by the hyperspectral camera 12 measuring the user U, similarly to the present estimation result.

Next, the controller 22 outputs, to the information terminal 10, information (state information) based on the determination result indicating the skin state of the user U determined by the determiner 22a (S23).

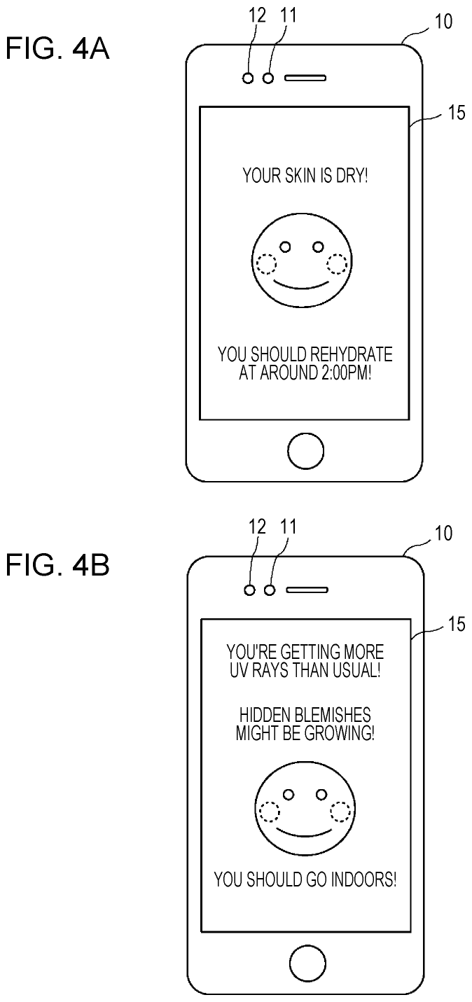

Next, the controller 13 of the information terminal 10 acquires the information from the server 20 and causes the information to be displayed through the display 15 (S13). The information displayed on the display 15 will be described with reference to FIGS. 4A and 4B. FIG. 4A is a diagram illustrating a first example of state information displayed on the information terminal 10 according to the present embodiment. FIG. 4B is a diagram illustrating a second example of state information displayed on the information terminal 10 according to the present embodiment.

FIG. 4A illustrates an example in which the display 15 displays information based on a determination result regarding the skin moisture content of the user U. As an example, information indicating that the skin is dry is displayed. For instance, information (dashed-line frames) indicating dry areas of the face of the user U may be displayed. At this time, the controller 13 may superimpose information indicating the dry areas onto face image data of the user U captured by the imaging camera 11, for example. In addition, a recommended action with respect to the determination result may also be displayed. For example, information indicating a recommendation to rehydrate around 2:00 p.m. may be displayed. Note that the dry areas and the recommended action are included in the information based on the determination result regarding the skin moisture content.

FIG. 4B illustrates an example in which the display 15 displays information based on a determination result regarding hidden blemishes in the skin of the user U. As an example, the user is being exposed to more ultraviolet light than usual, and consequently information indicating that there is a risk that hidden blemishes will increase is displayed. For instance, information (dashed-line frames) indicating areas of hidden blemishes on the face of the user U may be displayed.

At this time, the controller 13 may superimpose information indicating the areas of hidden blemishes onto face image data of the user U captured by the imaging camera 11, for example. In addition, a recommended action with respect to the determination result may also be displayed. For example, information indicating a recommendation to go indoors may be displayed. Note that the areas of hidden blemishes and the recommended action are included in the information based on the determination result regarding the hidden blemish conditions.

By checking the display illustrated in FIG. 4A or 4B, the user U can learn of his or her skin state at present and a recommended action. Note that the displays illustrated in FIGS. 4A and 4B may also be displayed at the same time. It is sufficient if the information displayed on the display 15 includes at least a determination result regarding the skin state at present. Specifically, in the example of FIG. 4A, it is sufficient if information indicating that the skin is dry is displayed. In the example of FIG. 4B, it is sufficient if information indicating that there are hidden blemishes is displayed.

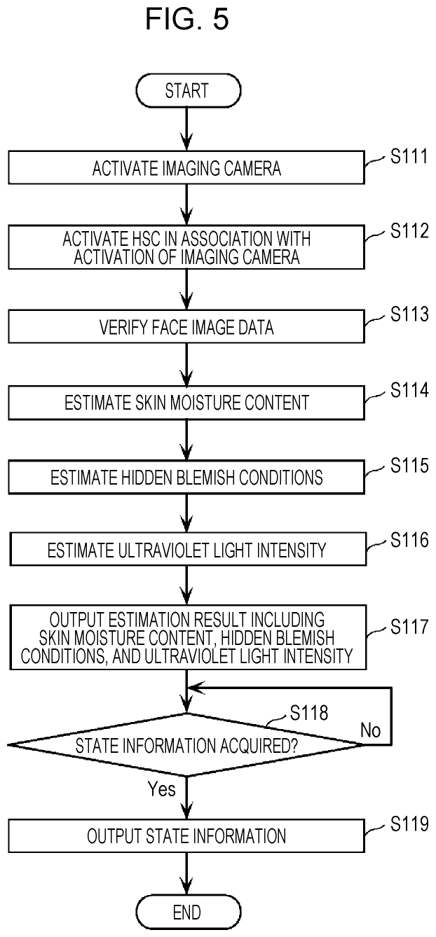

Next, operations by the information terminal 10 will be described with reference to FIG. 5. FIG. 5 is a flowchart illustrating operations by the information terminal 10 according to the present embodiment.

As illustrated in FIG. 5, the controller 13 of the information terminal 10 activates the imaging camera 11 (S111). For example, the controller 13 activates the imaging camera 11 upon detecting that the user U has performed a prescribed operation on the information terminal 10. The prescribed operation may be holding up the information terminal 10 in a sleep state or tapping the display 15, for example, but is not limited thereto. The prescribed operation may also be a voice-based operation, for example. Step S111 is an example of a second acquiring step.

When activated, the imaging camera 11 captures the face of the user U to generate face image data.

Next, the controller 13 activates the hyperspectral camera 12 (HSC) in association with the activation of the imaging camera 11 (S112). For example, the controller 13 may activate the hyperspectral camera 12 upon detecting that the prescribed operation has been performed on the information terminal 10.

When activated, the hyperspectral camera 12 measures at least the spectrum of the skin of the user U. In the present embodiment, the hyperspectral camera 12 measures the spectrum of the skin of the user U and the spectrum of sunlight when activated. With this arrangement, measurement data obtained by measuring the skin of the user U at present can be acquired. Step S112 is an example of a first acquiring step.

The measurement data is an example of a first measurement result.

Note that step S112 corresponds to step S11 illustrated in FIG. 3.

Next, the verifier 13b acquires face image data from the imaging camera 11 and compares the face image data to face image data stored in the storage 17 (S113). By comparing the face image data, the verifier 13b determines whether the user U is a registered user. Note that the method of verification by the verifier 13b is not particularly limited, and any known technology may be used.

Next, the analyzer 13a estimates the skin moisture content of the user U on the basis of the measurement data measured by the hyperspectral camera 12 (S114). For example, the analyzer 13a may estimate the skin moisture content of the user U on the basis of the spectrum of a band including a wavelength absorbed by water from among multiple bands. The wavelength absorbed by water may be a near-infrared or mid-infrared wavelength such as 750 nm, 970 nm, or 1450 nm, for example.

The analyzer 13a may estimate the skin moisture content of the user U on the basis of the spectrum of a band including a wavelength absorbed by water and the spectrum of a band including a wavelength weakly absorbed by water from among multiple bands. The wavelength weakly absorbed by water may be a near-infrared or mid-infrared wavelength such as 810 nm, 1160 nm, or 1300 nm, for example.

The analyzer 13a may estimate the skin moisture content of the user U on the basis of the spectrum of a band including a wavelength absorbed by water in the measurement data obtained by measuring the skin and the spectrum of a band including a wavelength absorbed by water in the measurement data obtained by measuring sunlight. In other words, the analyzer 13a may estimate the skin moisture content of the user U on the basis of the absorbance of skin at the wavelength absorbed by water. In this case, the analyzer 13a may calculate the absorption due to the skin moisture of the user U by calculating the difference between the spectrum of a band including a wavelength absorbed by water in the measurement data obtained by measuring the skin and the spectrum of a band including a wavelength absorbed by water in the measurement data obtained by measuring sunlight. With this arrangement, the influence due to ambient light can be removed, and the skin moisture content of the user U can be estimated more accurately. The analyzer 13a may estimate the skin moisture content of the user U according to any known method for estimating skin moisture content using a light spectrum.

Next, the analyzer 13a estimates the hidden blemish conditions of the user U on the basis of the measurement data measured by the hyperspectral camera 12 (S115). For example, the analyzer 13a may estimate the hidden blemish conditions of the user U on the basis of the spectrum of a band including a wavelength absorbed by melanin, which is the cause of hidden blemishes, from among multiple bands. The wavelength absorbed by melanin may be 660 nm or 880 nm, for example.

The analyzer 13a may estimate the hidden blemish conditions of the user U on the basis of the spectrum of a band including a wavelength absorbed by melanin and the spectrum of a band including a wavelength weakly absorbed by melanin from among multiple bands. The analyzer 13a may estimate the hidden blemish conditions of the user U on the basis of the spectrum of a band including a wavelength absorbed by melanin in the measurement data obtained by measuring the skin and the spectrum of a band including a wavelength absorbed by melanin in the measurement data obtained by measuring sunlight. In this case, the analyzer 13a may calculate the absorption due to melanin in the skin of the user U by calculating the difference between the spectrum of a band including a wavelength absorbed by melanin in the measurement data obtained by measuring the skin and the spectrum of a band including a wavelength absorbed by melanin in the measurement data obtained by measuring sunlight. With this arrangement, the influence due to ambient light can be removed, and the hidden blemish conditions of the user U can be estimated more accurately. The analyzer 13a may estimate the hidden blemish conditions of the user U according to any known method for estimating hidden blemish conditions using a light spectrum. Note that the hidden blemish conditions may be the presence or absence of a hidden blemish, the location of a hidden blemish, or the number of hidden blemishes.

Note that it is sufficient if at least one of step S114 or step S115 is performed. At least one of step S114 or step S115 is an example of a first estimating step.

Next, the analyzer 13a estimates the ultraviolet light intensity on the basis of the measurement data measured by the hyperspectral camera 12 (S116). For example, the analyzer 13a may estimate the ultraviolet light intensity that the user U is inferred to be exposed to on the basis of the spectrum of a band including a wavelength in the ultraviolet region from among multiple bands. The ultraviolet region is from 280 nm to 400 nm, for example. The analyzer 13a may estimate the ultraviolet light intensity that the user U is inferred to be exposed to on the basis of the spectrum of a band including a wavelength in the ultraviolet region in the measurement data obtained by measuring the skin and the spectrum of a band including a wavelength in the ultraviolet region in the measurement data obtained by measuring sunlight. The analyzer 13a may estimate the ultraviolet light intensity that the user U is exposed to or has absorbed according to any known method for estimating the ultraviolet light intensity that a person is exposed to or has absorbed using a light spectrum.

Note that step S116 is executed in the case where step S115 is performed. Step S116 is an example of a second estimating step.

Next, the controller 13 outputs an estimation result including the estimated skin moisture content, hidden blemish conditions, and ultraviolet light intensity to the server 20 through the communication device 14 (S117). The estimation result may also include identification information of the user U or identification information of the information terminal 10. In the present embodiment, the estimation result includes identification information of the user U. The estimation result may further include information related to the surrounding environment of the user U when the hyperspectral camera 12 measured the skin of the user U. The surrounding environment includes at least one of the temperature and humidity, the high temperature and low temperature, weather information, whether the user U is indoors or outdoors, or settings for equipment such as an air conditioner or a humidifier if the user U is indoors. If the information terminal 10 includes a temperature and humidity sensor, the temperature and humidity may be a measurement result from the temperature and humidity sensor, or the temperature and humidity may be acquired through a special-purpose application. The high temperature and low temperature, weather information, and the like are acquired from servers that manage each type of information, for example. If the information terminal 10 includes a GPS sensor, an indication of whether the user U is indoors or outdoors may be a result acquired from the GPS sensor, or an indication may be acquired from the user U through the input device 16. The settings for equipment may be acquired from a control device that centrally controls the equipment, acquired directly through communication with the equipment, or acquired from the user U through the input device 16. The estimation result may also include information related to biological information such as body temperature, heart rate, and blood pressure, and lifestyle information such as activity level, diet (nutrient intake) and hours of sleep. Biological information may be acquired from a device such as a smartphone or a wearable terminal equipped with a biosensor. Biological information may also be acquired from an application included in the smartphone. The conditions at the time of measurement included in the estimation result, such as the information indicating the surrounding environment, and biological information and lifestyle information about the user U, are collectively referred to as the “measurement conditions log”. By using the measurement conditions log, it is possible to make a determination according to the individual with consideration for the correlation with the measurement results at time points in the past, and improvements in the determination accuracy and learning efficiency can improve the accuracy of the estimation result.

Note that steps S114 to S117 correspond to step S12 illustrated in FIG. 3.

Next, the information terminal 10 determines whether state information based on a determination result obtained by the server 20 using the estimation result to determine the skin state of the user U has been acquired from the server 20 (S118). If state information has been acquired from the server 20 (S118, Yes), the information terminal 10 outputs the state information (S119). The controller 13 may superimpose information (for example, dashed-line frames) based on the determination result regarding the skin state included in the acquired state information onto face image data, and output state information including the superimposed face image data. With this arrangement, the controller 13 causes the display 15 to present a display like the one illustrated in FIG. 4A or 4B. Note that state information including face image data obtained by superimposing information based on the determination result onto face image data is an example of state information reflecting the determination result regarding the skin state. Reflecting herein includes superimposing, for example.

Note that the controller 13 may also, for example, display information indicating temporal changes in the estimation result estimated over a prescribed period. For example, the controller 13 may display temporal changes in the estimated skin moisture content over a single day with a line graph or the like. The controller 13 may also display information indicating temporal changes in the determination result regarding the skin state by the determiner 22a of the server 20. For example, the controller 13 may display information indicating whether the skin moisture content at present is higher than an average value of the skin moisture content estimated in the past.

The controller 13 successively stores the state information acquired from the server 20 in the storage 17, and therefore can display information indicating temporal changes in the determination result.

If state information has not been acquired from the server 20 (S118, No), the information terminal 10 returns to step S118 and stands by until state information is acquired.

Note that steps S118 and S119 correspond to step S13 illustrated in FIG. 3.

Note that the process from step S114 may be executed in the case where the user U is determined to be a registered user according to the verification of face image data by the verifier 13b. In other words, the process from step S114 may be executed in the case where the user U presently carrying the information terminal 10 is the regular user of the information terminal 10. The controller 13 may determine whether or not to execute the process from step S114 on the basis of the verification result in step S113.

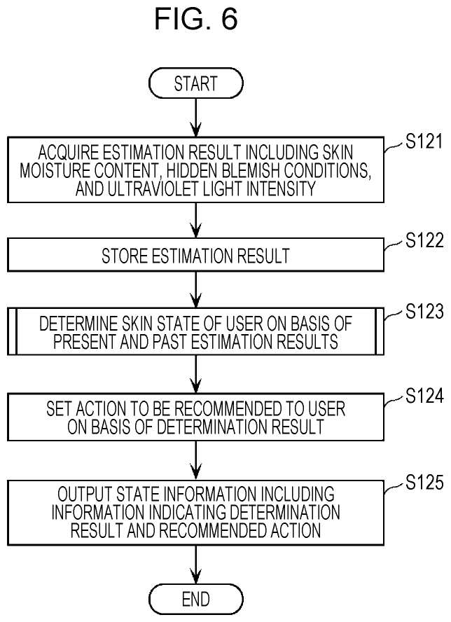

Next, operations by the server 20 will be described with reference to FIG. 6. FIG. 6 is a flowchart illustrating operations by the server 20 according to the present embodiment.

As illustrated in FIG. 6, the controller 22 of the server 20 acquires an estimation result including the skin moisture content, hidden blemish conditions, and ultraviolet light intensity through the communication device 21 (S121), and stores the acquired estimation result in the storage 23 (S122). The controller 22 stores the estimation result in the storage 23 as an estimation result corresponding to the user U on the basis of the identification information of the user U included in the estimation result. The controller 22 stores an estimation result in the storage 23 every time an estimation result is acquired. Accordingly, time series data of past estimation results for the user U is stored in the storage 23.

Note that steps S121 and S122 correspond to step S21 illustrated in FIG. 3.

Next, the determiner 22a of the controller 22 determines the skin state of the user U on the basis of present and past estimation results (S123). A method for determining the skin state will be described in detail later.

Note that step S123 corresponds to step S22 illustrated in FIG. 3.

Next, the determiner 22a sets an action to be recommended to the user U on the basis of the determination result (S124). For example, the determiner 22a sets an action to be recommended to the user U by using a table associating determination results with recommended actions. If the determination result includes an indication that the skin of the user U is dry, for example, the determiner 22a sets a recommended action such as rehydrating or applying a moisturizing cream. If the determination result includes an indication that hidden blemishes are forming in the skin of the user U, for example, the determiner 22a sets a recommended action such as going indoors or applying sunscreen.

Next, the controller 22 outputs state information including information indicating the determination result and the recommended action to the information terminal 10 (S125). The state information is an example of information based on the determination result.

Note that step S125 corresponds to step S23 illustrated in FIG. 3.

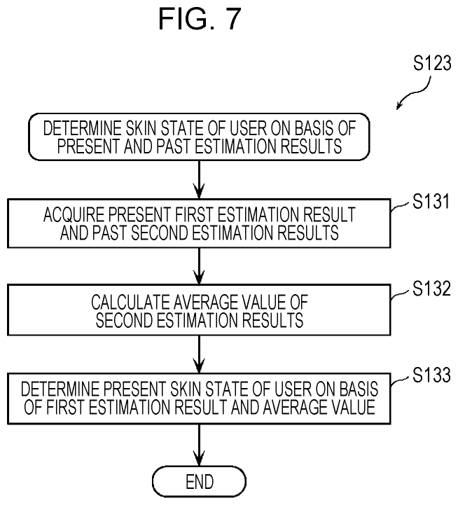

One example of a method for determining the skin state will be described with reference to FIGS. 7 to 10. FIG. 7 is a flowchart illustrating a first example of the operations (S123) for determining the skin state of a user illustrated in FIG. 6.

As illustrated in FIG. 7, the determiner 22a acquires the present estimation result (first estimation result) and past estimation results (second estimation results) by reading out the estimation results from the storage 23 (S131). For example, the determiner 22a may acquire all of the past second estimation results, the second estimation results in the same period, or the second estimation results acquired between the present and a prescribed amount of time before the present. The same period may be the same season (spring, summer, fall, or winter, for example), the same month, or a prescribed period before and/or after the present day, for example. The prescribed period may be one week or one month, for example.

Next, the determiner 22a calculates the average value of the read-out second estimation results (S132). In the case where the estimation results are related to the skin moisture content, in step 5132, the determiner 22a calculates the average value of the skin moisture content, for example. In the case where the estimation results are related to hidden blemish conditions, in step S132, the determiner 22a calculates the average value of the number of hidden blemishes, for example. The average value calculated here is different for each user.

Note that the determiner 22a may also use a weight average to calculate the average value of the second estimation results. For example, the determiner 22a may assign respective weights to the second estimation results such that a second estimation result acquired at a time point closer to the present is weighted more heavily. With this arrangement, large changes in the skin state occurring recently are detected more easily.

Next, the determiner 22a determines the present skin state of the user U on the basis of the first estimation result and the average value (S133). The determiner 22a may determine the present skin state of the user U by comparing the first estimation result to the average value. In the case where the estimation results are related to the skin moisture content, the determiner 22a determines that the skin is dry at present if the first estimation result is lower than the average value. In the case where the estimation results are related to hidden blemish conditions, the determiner 22a determines that hidden blemishes are forming or increasing at present if the first estimation result is higher than the average value.

Note that the determination method by the determiner 22a is not limited to the above, and may be another method if past second estimation results are used. For example, the determiner 22a may calculate a threshold value (less than the average value) of the skin moisture content for determining that the skin is dry on the basis of the second estimation results, and determine that the skin is dry if the first estimation result is lower than the calculated threshold value. As another example, the determiner 22a may calculate a threshold value (greater than the average value) of hidden blemish conditions for determining that hidden blemishes are increasing on the basis of the second estimation results, and determine that hidden blemishes are increasing if the first estimation result is higher than the calculated threshold value. The threshold value may be calculated by multiplying the average value by a prescribed coefficient or by accounting for variations in the second estimation results, for example.

The determiner 22a may determine the skin state on the basis of the position of the first estimation result in a distribution of the second estimation results. In the case where the estimation results are related to the skin moisture content, the determiner 22a may determine that the skin moisture content is appropriate if the first estimation result is positioned between the top 20% highest skin moisture content and the bottom 20% lowest skin moisture content among the second estimation results, and determine that the skin moisture content is inappropriate if the first estimation result is outside the above range, for example.

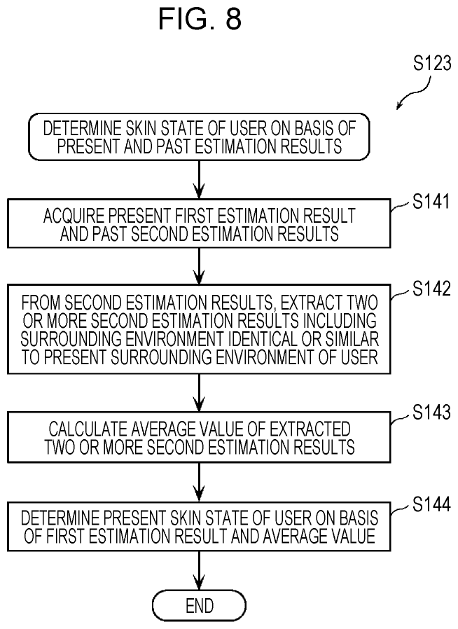

Next, another example of a method for determining the skin state will be described. FIG. 8 is a flowchart illustrating a second example of the operations (S123) for determining the skin state of a user illustrated in FIG. 6. FIG. 8 illustrates a process in the case where information related to the surrounding environment is included in the estimation results.

As illustrated in FIG. 8, the determiner 22a acquires the present estimation result (first estimation result) and past estimation results (second estimation results) by reading out the estimation results from the storage 23 (S141). Step S141 is similar to step S131 illustrated in FIG. 7, and therefore a description is omitted.

Next, the determiner 22a extracts, from among the second estimation results, two or more second estimation results including a surrounding environment that is identical or similar to the present surrounding environment of the user U (S142). The determiner 22a may be considered to extract, from among the second estimation results, two or more second estimation results that were acquired in a surrounding environment identical or similar to the present surrounding environment of the user U. On the basis of information (first surrounding environment information) indicating the surrounding environment included in the first estimation result and information (second surrounding environment information) indicating the surrounding environment included in each of the second estimation results, the determiner 22a extracts, from among the second estimation results, two or more second estimation results for which the second surrounding environment information is identical or similar to the first surrounding environment information. Note that similar means that the difference between the first surrounding environment information and the second surrounding environment information satisfies a prescribed condition, for example. For instance, taking the case where the surrounding environment is temperature as an example, the prescribed condition may be set on the basis of a correlation between the skin moisture content of the user U and the temperature, and may be a condition stipulating that the temperature difference is within 5 degrees, for example. It is beneficial to set the prescribed condition for each user U. Step S142 is not limited to information indicating the surrounding environment, and second estimation results may also be extracted on the basis of at least one correlation in the measurement conditions log indicating the conditions at the time of measurement included in the estimation results, such as biological information and lifestyle information about the user U.

Next, the determiner 22a determines the skin state of the user U on the basis of the first estimation result and the extracted two or more second estimation results. For example, the determiner 22a calculates the average value of the extracted two or more second estimation results (S143). Thereafter, the determiner 22a determines the present skin state of the user U on the basis of the first estimation result and the average value (S144).

Note that step S144 is similar to step S133 illustrated in FIG. 7, and therefore a description is omitted.

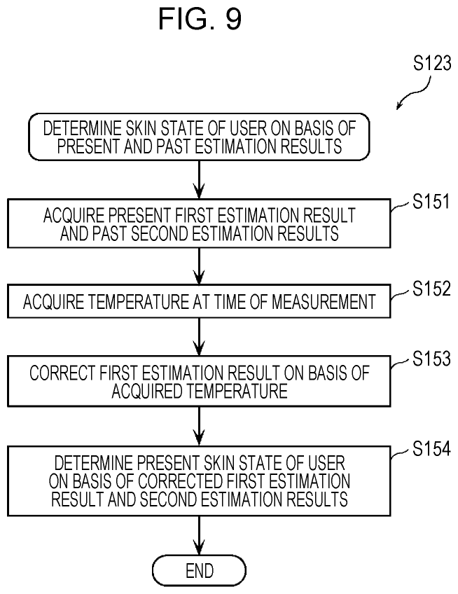

Next, yet another example of a method for determining the skin state will be described. FIG. 9 is a flowchart illustrating a third example of the operations (S123) for determining the skin state of a user illustrated in FIG. 6. FIG. 9 illustrates a process in the case where an estimation result is corrected according to the surrounding environment. FIG. 9 illustrates another example of a process in the case where information related to the surrounding environment is included in the estimation results. Specifically, FIG. 9 illustrates an example of a process in the case where at least one of the temperature or humidity around the user U at present is included in the estimation results. Hereinafter, the case where the temperature around the user U at present is included in the estimation results will be described.

In the following, the estimation results relate to the skin moisture content.

As illustrated in FIG. 9, the determiner 22a acquires the present estimation result (first estimation result) and past estimation results (second estimation results) by reading out the estimation results from the storage 23 (S151). Step S151 is similar to step S131 illustrated in FIG. 7, and therefore a description is omitted.

Next, the determiner 22a acquires the temperature (ambient temperature) at the time of the measurement taken by the hyperspectral camera 12 for acquiring the first estimation result (S152). For example, the determiner 22a acquires the temperature included in the first estimation result as the temperature at the time of measurement.

Next, the determiner 22a corrects the first estimation result on the basis of the acquired temperature (S153). For example, if the acquired temperature is different from a standard temperature, the determiner 22a corrects the first estimation result on the basis of the acquired temperature and the standard temperature. For example, the determiner 22a calculates the skin moisture content for the standard temperature on the basis of information indicating a correlation between the skin moisture content of the user U and the temperature pre-stored in the storage 23, and the skin moisture content for the acquired temperature. The information indicating a correlation may be a first-order approximation (regression line) indicating a linear regression model based on various temperatures and the skin moisture content of the user U at each of the temperatures, for example. With this arrangement, the skin moisture content can be corrected on the basis of a first-order approximation according to the user U him- or herself, and therefore can be corrected more accurately.

Next, the determiner 22a determines the present skin state of the user U on the basis of the corrected first estimation result and the second estimation results (S154). For example, the determiner 22a determines the present skin state of the user U on the basis of the corrected first estimation result and the average value of the second estimation results. The determiner 22a may determine the present skin state of the user U by comparing the corrected first estimation result to the average value. Note that the second estimation results here are values that have been pre-corrected to a moisture content for a standard temperature on the basis of the above correlation.

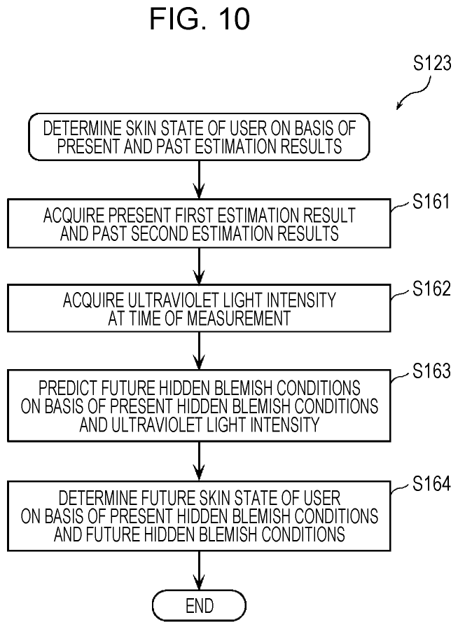

Next, yet another example of a method for determining the skin state will be described. FIG. 10 is a flowchart illustrating a fourth example of the operations (S123) for determining the skin state of a user illustrated in FIG. 6. FIG. 10 illustrates a process in the case where an estimation result is corrected according to the ultraviolet light intensity. FIG. 10 illustrates the case where hidden blemish conditions and the ultraviolet light intensity are included in the estimation results. Specifically, FIG. 10 illustrates a process in the case where hidden blemish conditions in the future are predicted on the basis of the ultraviolet light intensity.

As illustrated in FIG. 10, the determiner 22a acquires the present estimation result (first estimation result) and past estimation results (second estimation results) by reading out the estimation results from the storage 23 (S161). Step S161 is similar to step S131 illustrated in FIG. 7, and therefore a description is omitted.

Next, the determiner 22a acquires the ultraviolet light intensity at the time of the measurement taken by the hyperspectral camera 12 for acquiring the first estimation result (S162). For example, the determiner 22a acquires the ultraviolet light intensity included in the first estimation result as the ultraviolet light intensity at the time of measurement.

Next, the determiner 22a predicts future hidden blemish conditions on the basis of the present hidden blemish conditions and the ultraviolet light intensity (S163). The determiner 22a may predict future hidden blemish conditions by using the present hidden blemish conditions and a table associating ultraviolet light intensities with future hidden blemish conditions, or by using a trained model that accepts the present hidden blemish conditions and the ultraviolet light intensity as input and outputs future hidden blemish conditions. The table is generated on the basis of past second estimation results about the user and a degree of change (for example, an amount of increase or decrease) in hidden blemishes of the user in the future from that time. The trained model is generated by using machine learning that treats second estimation results about the user and ultraviolet light intensities as training data, and treats amounts of change in hidden blemishes of the user in the future from that time as ground truth data.

In this way, the determiner 22a determines the skin state of the user U in the first estimation result by using a table or a trained model generated on the basis of second estimation results, for example. The ultraviolet light intensity and degree of change in hidden blemishes are different for each user U. For this reason, it is beneficial to generate the table or trained model for each user U individually or for users U with similar attributes. In this case, the determiner 22a may be considered to predict future hidden blemish conditions on the basis of the present hidden blemish conditions of the user and a correlation between the ultraviolet light intensity and the degree of change in hidden blemishes of the user U. Note that users with similar attributes may be, for example, users in the same age group, users of the same sex, or users living in the same geographical area. Information indicating a correlation between the ultraviolet light intensity and the degree of change in hidden blemishes of the user U is pre-stored in the storage 23.

Note that future hidden blemish conditions may be, for example, an increase of 20% in hidden blemishes five days later. Five days later is one example of a third time point, for instance.

Next, the determiner 22a determines the future skin state of the user U on the basis of the present hidden blemish conditions and the future hidden blemish conditions (S164). Here, the determiner 22a determines how the future hidden blemish conditions will change in relation to the present hidden blemish conditions. In other words, the determiner 22a determines the skin state of the user U at a third time point.

However, some of the above steps do not have to be executed. For example, in the case where machine learning is used, the skin state of the user may be determined by analyzing the measured spectrum using a trained model, without executing the step of calculating an estimation result from the spectrum. For example, the determiner 22a determines the skin state of the user U in the first measurement result by using a trained model generated on the basis of second estimation results. The ultraviolet light intensity and degree of change in hidden blemishes are different for each user U. For this reason, it is beneficial to generate the trained model for each user U individually or for users U with similar attributes. In this case, the determiner 22a may be considered to predict future hidden blemish conditions on the basis of the present hidden blemish conditions of the user U and a correlation between the ultraviolet light intensity and the degree of change in hidden blemishes of the user U.

In this way, the determiner 22a may acquire a determination result regarding the skin state of the user U at present by inputting the first measurement result into a trained model that has been trained to accept second measurement results as input and output a determination result regarding the skin state of the user U. The acquisition of a determination result regarding the skin state by the determiner 22a is one example of a determining step.

Such a trained model is generated by using machine learning that treats measurement results (for example, spectra) measured by the hyperspectral camera 12 as training data, and treats the skin states at the time of the measurement results as ground truth data. The trained model is generated by deep learning using a neural network, for example.

The trained model may be trained to accept second measurement results including a measurement conditions log at each of the second time points as input and output a determination result regarding the skin state of the user U. The determiner 22a may also acquire a determination result regarding the skin state of the user at a first time point by inputting, into the trained model, the first measurement result including the measurement conditions log for the user at the first time point.

Such a trained model is generated by using machine learning that treats measurement results (for example, second measurement results) including a measurement conditions as training data, and treats the skin states at the time of the measurement results as ground truth data.

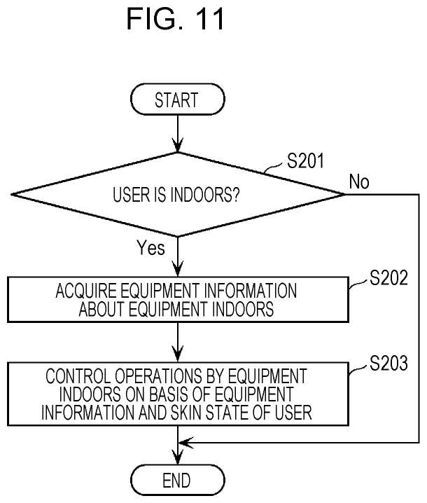

Next, the control of equipment indoors by the server 20 in the case where it is determined that the skin state is degraded, such as that the skin is dry or that hidden blemishes are increasing, will be described with reference to FIG. 11. FIG. 11 is a flowchart illustrating operations for controlling equipment indoors by the server 20 according to the present embodiment. FIG. 11 is executed after the determination in step S123 of FIG. 6, for example.

As illustrated in FIG. 11, the controller 22 determines whether the user U is indoors at present (S201). For example, the controller 22 executes the determination in step S201 in the case where the determiner 22a has determined that the skin state is degraded, such as that the skin is dry or that hidden blemishes are increasing. The controller 22 determines whether the user U is indoors on the basis of location information (such as latitude, longitude, and altitude, for example) about the user U acquired from the information terminal 10, for example. Step S201 is an example of a second determining step.

Next, if the user U is indoors (S201, Yes), the controller 22 acquires equipment information about equipment indoors (S202), and controls operations by the equipment indoors on the basis of the acquired equipment information and the skin state of the user U (S203). Step S203 is an example of a controlling step.

The equipment is electrical equipment installed indoors, such as an air conditioner, a humidifier, a dehumidifier, a heater, or a motorized curtain, for example. For instance, in the case where the equipment is a humidifier and the skin of the user U is dry, the controller 22 acquires equipment information including the presently set humidity from the humidifier. Additionally, the controller 22 determines whether the set humidity is a value suited to the present skin state of the user U, and if the set humidity is not a value suited to the skin state of the user U, the controller 22 raises the set humidity of the humidifier. If the humidifier is not presently operating, the controller 22 causes the humidifier to start operating. Note that the controller 22 may also control the set humidity of the humidifier on the basis of the present humidity in the space where the user U is present rather than the set humidity. The equipment is communicably connected to the server 20 over the network N.

As above, the determiner 22a according to the present embodiment uses the present estimation result and past estimation results to determine the present skin state of the user U based on the spectrum measured by the hyperspectral camera 12. With this arrangement, a determination can be made according to the properties of the skin of the user U, and the skin state of the user U can be determined more accurately.

Note that although the embodiment above describes an example in which the skin state of the user includes at least one of the skin moisture content or hidden blemish conditions, the skin state of the user is not limited thereto. The skin state of the user may also be at least one of a quantity of resident microbiota, a loss of skincare product or a loss of sunscreen, the cleansing state of a skincare product, a salt content (for example, the salt content of sweat), the state of the teeth or the tongue, inflammation conditions such as acne, the conditions of dark areas around the eyes, or conditions related to personal grooming such as overgrown nose hair.

The quantity of resident microbiota is estimated on the basis of the spectrum of a band including wavelengths absorbed by resident microbiota, for example. Resident microbiota refers to bacteria that normally live on the skin, such as propionibacterium acnes, for example. Resident microbiota absorb the 540 nm wavelength, for example. The server 20 can determine whether resident microbiota are increasing on the basis of the present quantity of resident microbiota and past quantities (at different time points) of resident microbiota. An action regarding whether to wash one's face can be recommended as a recommended action.

The server 20 can determine a loss of skincare product, a loss of sunscreen, or the cleansing state of a skincare product on the basis of the present state of loss of a skincare product or sunscreen and a past state of the skincare product or sunscreen (for example, the state immediately after the product was applied to the skin).

The timing at which to touch up one's face, the reapplication of sunscreen, the reapplication of a cleanser, or the like can be recommended as a recommended action.

The server 20 may also estimate the state of health of the user according to dirty teeth, incomplete brushing, the state of the tongue, or the like. The server 20 may also determine inflammation or swelling conditions such as acne. The server 20 may determine a rested state, a nutritional state, or the like according to the types of dark areas around the eyes. If a nose hair is detected, the server 20 may also determine that the nose hair is overgrown.

### Other Embodiments

The above describes a skin state determination method and the like according to one or more aspects on the basis of an embodiment, but the present disclosure is not limited to the embodiment. Embodiments obtained by applying various modifications that may occur to a person skilled in the art as well as embodiments constructed by combining the structural elements in different embodiments may also be included within the scope of the one or more exemplary embodiments of the present disclosure, insofar as such embodiments do not depart from the gist of the present disclosure.

For example, the above embodiment describes an example in which the information terminal includes a hyperspectral camera, but the information terminal is not limited thereto. The information terminal may include a multispectral camera or an ultra-spectral camera in place of a hyperspectral camera. A multispectral camera is a spectral camera that can acquire spectra in a maximum of appropriately 10 bands, while an ultra-spectral camera is a spectral camera that can acquire spectra in 1000 bands or more. Even if the information terminal includes a multispectral camera or an ultra-spectral camera, the obtained effects of the present disclosure are unchanged. A multispectral camera and an ultra-spectral camera are examples of a spectral camera.

The above embodiment and the like describe an example in which the controller of the information terminal activates the hyperspectral camera in association with the activation of the imaging camera, but the configuration is not limited thereto. The hyperspectral camera may also be controlled irrespectively of the activation of the imaging camera. For example, the controller may also activate the hyperspectral camera at prescribed time intervals or at a prescribed time, or activate the hyperspectral camera in response to user input provided through the input device.

The above embodiment describes an example in which the skin moisture content and hidden blemish conditions are obtained by measuring the face of the user, but the configuration is not limited thereto. The skin moisture content and hidden blemish conditions may also be obtained by measuring the skin of the user on a part of the body than the face. For example, the skin moisture content and hidden blemish conditions may also be obtained by measuring the user's neck, hand, or the like.

The information terminal in the above embodiment may also include a light source for measuring the skin moisture content, for example. The light source may irradiate the skin with light including a near-infrared or mid-infrared wavelength absorbed by water, and the hyperspectral camera may measure the intensity of returning light that is reflected by the skin, for example.

The above embodiment describes an example in which the information terminal uses light with a visible-light or near-infrared wavelength to estimate hidden blemish conditions, but the configuration is not limited thereto. The information terminal may also use light with a near-ultraviolet wavelength to estimate hidden blemish conditions.

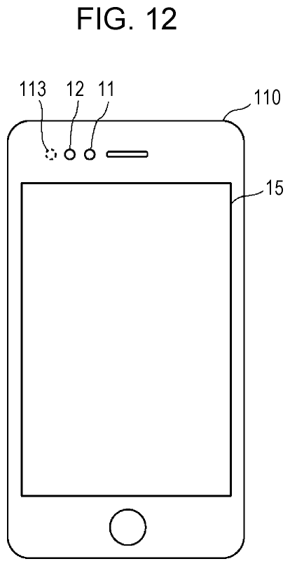

The above embodiment describes an example in which the information terminal is provided with the hyperspectral camera on the same surface as the imaging camera, but another hyperspectral camera may also be provided on the surface on the opposite side. FIG. 12 is a diagram illustrating a configuration of an information terminal 110 according to another embodiment. As illustrated in FIG. 12, the information terminal 110 includes an imaging camera 11 and hyperspectral cameras 12 and 113, for example. The hyperspectral camera 113 is provided on the surface on the opposite side from the surface of the information terminal 110 on which the imaging camera 11 and the hyperspectral camera 12 are provided. With this arrangement, the spectrum of the skin of the user U can be measured by the hyperspectral camera 12 and the spectrum of sunlight can be measured by the hyperspectral camera 113. The hyperspectral camera 113 may be activated when sunlight is measurable depending on the direction of the information terminal 110 and the position of the sun, for example.

The order in which the steps in the flowcharts are executed is an exemplary order for describing the present disclosure specifically, and the order may also be different from the above. Some of the steps may be executed at the same time as (in parallel with) other steps, and not all of the steps have to be executed.

The divisions of the function blocks in the block diagrams are an example, and it is also possible for multiple function blocks to be achieved as a single function block, for a single function block to be subdivided, or for some functions to be moved to another function block. The functions of multiple function blocks having similar functions may be processed by the same hardware or software in parallel or by time division.

A skin state determination system may also be achieved by a single device. Although an example is described in which the information terminal and the server are each achieved by a single device, each of the information terminal and the server may also be achieved by multiple devices. For example, in the case where the information terminal is achieved by multiple devices, the components included in the information terminal may be distributed among the multiple devices in any way. For example, in the case where the server is achieved by multiple devices, the components included in the server may be distributed among the multiple devices in any way. The components provided in the skin state determination system may be distributed in any way among the devices provided in the skin state determination system. For example, at least one component included in the information terminal may be included in the server. For example, the server may include the analyzer.

The method of communication between the devices provided in the skin state determination system according to the above embodiment is not particularly limited, and may be wireless communication or wired communication.

Devices may also communicate with each other through a combination of wireless and wired communication.

Some or all of the components provided in the skin state determination system in the above embodiment may also be configured as a single system large-scale integration (LSI) chip.

A system LSI chip is a multi-function LSI chip fabricated by integrating multiple processing units onto a single chip, and specifically is a computer system including a microprocessor, read-only memory (ROM), random access memory (RAM), and the like. A computer program is stored in the ROM. The microprocessor operates in accordance with the computer program and thereby achieves the functions of the system LSI chip.

One aspect of the present disclosure may also be a computer program causing a computer to execute the characteristic steps included in the skin state determination method. For example, the program may be a program to be executed by the computer. One aspect of the present disclosure may also be a non-transitory computer-readable recording medium on which such a program is recorded. For example, such a program may be recorded onto the recording medium and circulated or distributed. For example, by installing the circulated program onto a device including another processor and causing the processor to execute the program, it is possible to cause the device to perform the processes described above.

The present disclosure is broadly usable in devices that determine the skin state of a user and the like.

What is claimed is:

1. A skin state determination method comprising:
acquiring a first measurement result including data obtained by a spectral camera measuring the skin of a user at a first time point, second time points before the first time point being different from each other;
estimating, on a basis of the first measurement result, a first estimation result including at least one of a skin moisture content or hidden blemish conditions of the user at the first time point; and
determining a skin state of the user on a basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

2. The skin state determination method according to claim 1, wherein
in the determining, two or more second estimation results including measurement conditions logs are extracted as the second estimation results, and the determination is made on a basis of the extracted two or more second estimation results, a measurement conditions log indicating measurement conditions at the first time point being identical or similar to each of the measurement conditions logs.

3. The skin state determination method according to claim 1, wherein
the first estimation result includes at least the hidden blemish conditions,
the skin state determination method further comprises estimating an ultraviolet light intensity at the first time point on a basis of the first estimation result, and
in the determining, the hidden blemish conditions of the user at a third time point after the first time point are estimated on a basis of the hidden blemish conditions of the user based on the first estimation result and the ultraviolet light intensity, and a determination for the third time point is made.

4. The skin state determination method according to claim 1, further comprising:
acquiring image data obtained by an imaging camera capturing the skin of the user; and
outputting state information reflecting, in the image data, a determination result regarding the skin state of the user.

5. The skin state determination method according to claim 4, wherein
the state information includes information indicating an action to be recommended to the user based on the determination result.

6. The skin state determination method according to claim 4, wherein
the spectral camera and the imaging camera operate synchronously.

7. The skin state determination method according to claim 1, further comprising:
determining whether the user is indoors at the first time point; and
if the user is determined to be indoors, controlling operations by equipment indoors according to the skin state of the user.

8. The skin state determination method according to claim 1, wherein
the first measurement result includes data obtained by the spectral camera measuring sunlight at the first time point, and
in the estimating, the difference between the data obtained by measuring the skin of the user and the data obtained by measuring the sunlight is taken.

9. A skin state determination method comprising:
acquiring a first estimation result including at least one of a skin moisture content or hidden blemish conditions of a user at a first time point, the first estimation result being based on a first measurement result including data obtained by a spectral camera measuring the skin of the user at the first time point, second time points before the first time point being different from each other; and
determining a skin state of the user on a basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

10. A skin state determination method comprising:
acquiring a first measurement result including data obtained by a spectral camera measuring the skin of a user at a first time point, second time points before the first time point being different from each other;
acquiring second measurement results including data obtained by the spectral camera measuring the skin of the user at each of the second time points; and
acquiring a determination result regarding a skin state of the user at the first time point by inputting the first measurement result into a trained model that has been trained to accept the second measurement results as input and output a determination result regarding the skin state of the user.

11. The skin state determination method according to claim 10, wherein
the trained model is trained to accept the second measurement results including measurement conditions logs at each of the second time points as input and output a determination result regarding the skin state of the user, and
in the acquiring of a determination result, a determination result regarding the skin state of the user at the first time point is acquired by inputting, into the trained model, the first measurement result including a measurement conditions log for the user at the first time point.

12. A skin state determination system comprising:
an acquirer that acquires a first measurement result including data obtained by a spectral camera measuring the skin of a user at a first time point;
an estimator that produces, on a basis of the first measurement result, a first estimation result including at least one of a skin moisture content or hidden blemish conditions of the user at the first time point; and
a determiner that determines a skin state of the user on a basis of the first estimation result and second estimation results based on second measurement results, each of the second estimation results including at least one of a skin moisture content or hidden blemish conditions of the user based on each of the second measurement results including data obtained by the spectral camera measuring the skin of the user at each of the second time points.

13. A skin state determination method comprising:
acquiring a first measurement result including a first data item about the skin of a user obtained by a spectral camera measuring the skin of the user at a first time point;
estimating, on a basis of the first measurement result, a first estimation result including at least one of a skin moisture content of the user or hidden blemish conditions of the user at the first time point; and
determining a skin state of the user on a basis of the first estimation result and the second estimation results, wherein
the second measurement results include second data items about the skin of the user obtained by the spectral camera measuring the skin of the user at second time points,
one or more third measurement results include third data items about the skin of the user, the third data items being extracted from among the second data items and obtained by the spectral camera measuring the skin of the user at one or more third time points,
the second estimation results include at least one of one or more skin moisture contents of the user or one or more hidden blemish conditions of the user at the one or more third time points estimated on a basis of the one or more third data items,
the second time points are before the first time point and the second time points are different from one another,
the first measurement result includes a first measurement log indicating conditions when the first data item was measured, and the first measurement log corresponds to the first data item,
the second measurement results include second measurement logs indicating conditions when the second data items were measured, and the second measurement logs correspond one-to-one to the second data items,
one or more logs are extracted from the second measurement logs, and each of the extracted one or more logs is identical or similar to the first measurement log,
one or more data items are extracted from the second data items, and the extracted one or more data items correspond one-to-one to the extracted one or more logs, and
the extracted one or more data items are the one or more third data items.
